(19) United States
(12) Patent Application Publication
(10) Pub. No.: US 2023/0293451 A1
(43) Pub. Date: Sep. 21, 2023

(54) STIMULATION OF HAIR GROWTH

(71) Applicant: The General Hospital Corporation, Boston, MA (US)

(72) Inventors: David E. Fisher, Newton, MA (US); Xu Wu, Lexington, MA (US); Mack Su, Boston, MA (US); Tal Erlich, Brookline, MA (US)

(21) Appl. No.: 17/791,770

(22) PCT Filed: Jan. 8, 2021
(86) PCT No.: PCT/US2021/012737

§ 371 Date: Jul. 8, 2022

Related U.S. Application Data
(60) Provisional application No. 62/959,594, filed on Jan. 10, 2020.

(51) Int. Cl. A61K 31/122; C07C 49/457; A61K 45/06; A61K 31/047; A61K 31/506; A61K 31/58; A61K 9/00; A61P 17/14; A61P 29/00

(52) U.S. Cl. CPC A61K 31/122; C07C 49/457; A61K 45/06; A61K 31/047; A61K 31/506; A61K 31/58; A61K 9/0014; A61P 17/14; A61P 29/00

(57) ABSTRACT

Compositions and methods that can increase or stimulate hair growth, as well as novel chemicals that affect hair growth and hair loss.

### CLAIM OF PRIORITY

This application claims the benefit of U.S. Provisional Patent Application Ser. No. 62/959,594, filed on Jan. 10, 2020. The entire contents of the foregoing are hereby incorporated by reference.

### TECHNICAL FIELD

The present disclosure relates to hair care compositions and methods that can stimulate or accelerate the growth of hair. The present disclosure also relates to the new chemical compound, squaric acid di(trifluromethyl)propyl ester (CF3-SADBE), and methods of use thereof.

### BACKGROUND

Most mammals are covered with hair. Hairloss (also known as alopecia or baldness) is a condition that involves a complete or partial loss of hair growth and affects a significant proportion of the human population. While desirable for some, the loss of hair can be a cause for concern for those who wish to preserve a more youthful appearance. Currently used treatments for baldness have met with little success and involve significant side effects to the patient, including anti-androgen related toxicities or inflammation of the dermis.

### SUMMARY

The present disclosure is based, at least in part, on the development of a new compound. The present disclosure is also based on the discovery of a new compound that can be useful in stimulating, increasing, thickening, or accelerating hair growth. The disclosure is also based, at least in part, on new treatment methods to inhibit, reduce, delay or treat the loss of hair. As described herein, the loss of hair can be reduced, inhibited, delayed, or treated by applying to the scalp and/or hair follicles effective amounts of the compositions and formulations described herein. As described herein, hair growth can be stimulated, increased, thickened, or accelerated by applying to the scalp and/or hair follicles effective amounts of the compositions and formulations described herein. In some cases, the compositions described herein can comprise squaric acid di(trifluromethyl)propyl ester (CF3-SADBE) or a physiologically acceptable salt thereof. In some cases, the compositions comprise SADBE or diphenylcyclopropenone (DPCP).

The compositions and methods described here can be useful in stimulating, increasing, or accelerating hair growth. In some cases, the compositions and methods described herein demonstrate less inflammation than known treatments.

In one aspect, the present disclosure provides for a compound of formula (1):

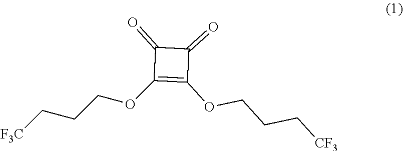 [SMILES: O=c1c(OCCCC(F)(F)F)c(OCCCC(F)(F)F)c1=O]

or squaric acid di(trifluromethyl)propyl ester (CF3-SADBE). In some embodiments, the compound is a physiological salt of CF3-SADBE. In another aspect, the present disclosure provides for a pharmaceutical composition comprising, consisting of, or consisting essentially of the compound of formula (1), and a pharmaceutically acceptable carrier. In some embodiments, the composition also includes one or more anti-inflammatory agents. The anti-inflammatory agents can be glucocorticoid agents, calcipotriol, an immunosuppressive agent, and/or antihistamines. In some cases, the composition can also include an agent selected from minoxidil, finasteride, SADBE, DPCP, and combinations thereof. In some embodiments, the composition is formulated for topical or subcutaneous administration.

In another aspect, the disclosure provides for a pharmaceutical composition for increasing hair growth in a mammal in need thereof comprising, consisting of, or consisting essentially of an effective amount of the compound of formula (1) and a pharmaceutically or dermatologically acceptable carrier.

In another aspect, the disclosure provides for a hair care composition for increasing hair growth in a mammal in need thereof comprising, consisting of, or consisting essentially of an effective amount of the compound in formula (1) and a dermatologically acceptable carrier.

In another aspect, the disclosure provides for a method of increasing the percentage of hair follicles in anagen phase and decreased percentage of hair follicles in telogen phase in a subject, the method comprising, consisting of, or consisting essentially of administering to a subject in need thereof an effective amount of the compound of formula (1). In some embodiments the subject has alopecia.

In another aspect, the disclosure provides for a method of treating hair loss in a subject, the method comprising, consisting of, or consisting essentially of administering a therapeutically effective amount of the compound of formula (1) to a subject in need thereof. In some cases, the hair loss is associated with alopecia.

In another aspect, the disclosure provides for each a method of stimulating hair growth, a method of increasing hair growth, and a method for inhibiting hair loss, each method comprising, consisting of, or consisting essentially of administering a therapeutically effective amount of a compound of formula (1).

In another aspect, the disclosure provides for a method for increasing hair growth in a subject, the method comprising, consisting of, or consisting essentially of topically applying an effective amount of a formulation to hair follicles and/or to skin overlying hair follicles of the subject, wherein the formulation comprises, consists of, or consists essentially of the compound of formula (1) and an acceptable carrier, and the formulation is applied one, two, three, or more times. In another aspect, the disclosure provides for each a method of promoting transition of hair follicles into anagen phase, a method of thickening hair, and a method of reducing hair thinning, each method comprising, consisting of, or consisting essentially of administering a composition comprising, consisting of, or consisting essentially of an effective amount of formula (1).

In another aspect, the disclosure provides for a pharmaceutical composition for increasing hair growth in a mammal in need thereof comprising, consisting of, or consisting essentially of an effective amount of SADBE and/or DPCP and a pharmaceutically or dermatologically acceptable carrier, wherein the mammal does not have an autoimmune disease.

In another aspect, the disclosure provides for a method of promoting transition of hair follicles from telogen phase to anagen phase, the method comprising, consisting of, or consisting essentially of administering to a subject in need thereof an effective amount of SADBE and/or DPCP, wherein the subject does not have an autoimmune disease. In some embodiments, the subject has alopecia.

In another aspect, the disclosure provides for a method of treating hair loss in a subject, the method comprising, consisting of, or consisting essentially of administering a therapeutically effective amount of SADBE and/or DPCP, wherein the hair loss is not associated with an autoimmune disorder. In some embodiments, the hair loss is associated with non-autoimmune alopecia.

In another aspect, the disclosure provides for each a method of stimulating hair growth in a subject in need thereof, a method of increasing hair growth in a subject in need thereof, and a method of inhibiting hair loss in a subject in need thereof, each method comprising, consisting of, or consisting essentially of administering a therapeutically effective amount of a SADBE and/or DPCP, wherein the subject does not have an autoimmune disease.

In another aspect, the disclosure provides for a method of increasing hair growth in a subject in need thereof, the method comprising, consisting of, or consisting essentially of topically applying an effective amount of a formulation to hair follicles and/or to the skin overlying hair follicles, wherein the formulation comprises, consists of, or consists essentially of SADBE and/or DPCP and an acceptable carrier, the formulation is applied one, two, three, or more times, and the subject does not have hair loss associated with an autoimmune disease.

In another aspect, the disclosure provides for each a method of promoting transition of hair follicles from telogen phase into anagen phase in a subject in need thereof, a method of thickening hair in a subject in need thereof, and a method of reducing hair thinning in a subject in need thereof, each method comprising, consisting of, or consisting essentially of administering a composition comprising, consisting of, or consisting essentially of an effective amount of SADBE or DPCP, wherein the subject does not have hair loss associated with an autoimmune disease.

In some embodiments of all aspects, alopecia can include acne keloidalis (e.g., folliculitis keloidalis, acne keloidalis nuchae), alopecia areata (auto-immune alopecia; e.g., ophiasis), alopecia totalis, alopecia universalis, anagen effluvium, androgenetic alopecia (e.g., male and female pattern hair loss), Brocq's alopecia, central centrifugal cicatricial alopecia, cicatricial (scarring) alopecias (e.g., primary cicatricial alopecia), congenital alopecia, congenital atrichia, diffuse alopecia areata, discoid (lesions) lupus erythematosus (DLE), dissecting cellulitis (e.g., perifolliculitis, abscedens et suffodiens), “end-stage” or “burnt-out”cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris (e.g., diffuse), lipedematous alopecia (lipedematous scalp), male pattern hair loss (e.g., bitemporal), non-cicatricial (non-scarring) alopecias, psoriasis, psoriasiform alopecia, pressure (postoperative) alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis. In some embodiments of all aspects, alopecia associate with an autoimmune disease includes alopecia areata. In some embodiments of all aspects, alopecia not associatde with an autoimmune disease (e.g., non-autoimmune alopecia) includes acne keloidalis (e.g., folliculitis keloidalis, acne keloidalis nuchae), alopecia totalis, alopecia universalis, anagen effluvium, androgenetic alopecia (e.g., male and female pattern hair loss), Brocq's alopecia, central centrifugal cicatricial alopecia, cicatricial (scarring) alopecias (e.g., primary cicatricial alopecia), congenital alopecia, congenital atrichia, diffuse alopecia areata, discoid (lesions) lupus erythematosus (DLE), dissecting cellulitis (e.g., perifolliculitis, abscedens et suffodiens), “end-stage” or “burnt-out”cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris (e.g., diffuse), lipedematous alopecia (lipedematous scalp), male pattern hair loss (e.g., bitemporal), non-cicatricial (non-scarring) alopecias, psoriasis, psoriasiform alopecia, pressure (postoperative) alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis.

In some embodiments of all aspects, the compound, formulation, or composition is administered to the skin, hair, or scalp of a mammal, patient, or subject in need thereof.

In some embodiments of all aspects, the methods described herein also include administering an agent selected from the group consisting of minoxidil, finasteride, DPCP, SADBE, and combinations thereof.

In some embodiments, the methods described herein increase hair growth by at least approximately 1, 2, 3, 4, 5, 6, 7, 8, 9, 10, 15, 20, 25, or 30%.

In some embodiments of all aspects, the methods described herein also include administering one or more steroids.

In some embodiments of all aspects, the methods described herein also include administering one or more agents to decrease sensitivity of the scalp or skin of the mammal (e.g., patient or subject) in need thereof.

In some embodiments of all aspects, the methods described herein also include administering one or more agents to modulate inflammation.

In some embodiments of all aspects, the formulations described herein also include one or more of an aqueous gel, alcoholic gel, ointment, oil, alcoholic or aqueous fluid, water-in-oil emulsion, oil-in-water emulsion, and water-in-silicone emulsion.

In some embodiments of all aspects, the formulations described herein also include an acceptable accessory ingredient selected from the group consisting of xanthan gum, glycerin, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance, and water.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIGS. 1A-D demonstrate the induction of anagen and hair growth in mice using topical application of SADBE and DPCP. FIG 1A shows the molecular structures of SADBE and DPCP. Both are contact sensitizers (haptens). FIG. 1B is a schematic illustrating the timeline for topical administration of haptens to mouse skin with hair follicles arrested in telogen. FIG. 1C is a collection of pictures of mice topically administered with haptens. Red circles show the treated flanks demonstrating anagen and hair growth. Green circles show untreated flanks demonstrating telogen. Treatment with contact sensitizers frequently produces cutaneous inflammation (red “*”). FIG. 1D is a graph showing the relative anagen induction measured by increased skin pigmentation intensity (a marker of anagen) on the treated side as a percent of the baseline (relative to) the untreated side.

FIGS. 2A-C show novel compounds that induce anagen and hair growth. FIG. 2A shows the molecular structures of SADBE derivatives that were synthesized and demonstrated improved anagen induction and/or decreased inflammation. FIG. 2B shows images of mice administered with SADBE, CF3-SADBE, and CC1-SADBE. CF3-SADBE appeared to induce anagen and hair growth at least as efficiently as SADBE with less inflammation. CC1-SABDE neither produced inflammation nor anagen and hair growth. Red circles show treated flanks demonstrating anagen and hair growth. Green circles show untreated flanks demonstrating telogen. Dotted red circle shows flank treated with CC1-SADBE with hair remaining in telogen. (red “*”) marks inflammation in the SADBE treated flank. FIG. 2C is a graph showing relative anagen induction for SADBE and its derivatives.

FIGS. 3A-B show CF3-SADBE efficiently induces anagen and hair growth with less inflammation than SADBE. FIG. 3A are images showing that treatment with 100 mM SADBE induces anagen with substantial inflammation by day 14 (blue “*”, blue circle), whereas the same dose of CF3-SADBE induces anagen with much less visible inflammation (red circle). Robust hair growth is observed by day 17 on the treated flank with both treatments. FIG. 3B is a graph showing relative anagen induction as a percent of the baseline untreated side measured over time for both treatments.

FIGS. 4A-C are images showing that CF3-SADBE treatment induces resting telogen hair follicles to enter anagen. FIG. 4A is an image showing gross appearance of a mouse treated with 100 mM CF3-SADBE on the left flank and untreated on the right flank. FIG. 4B is a haemotoxylin and eosin (H&E) staining of CF3-SADBE treated skin showing hair follicles in anagen (red “*”) where as FIG. 4C shows the hair follicles in the untreated skin remain in telogen (green “*”).

FIG. 5 are images showing CF3-SADBE induced anagen and hair growth earlier and with less inflammation than SADBE. Treatment with CF3-SADBE induces visible anagen by day 10, whereas SADBE does not (purple arrow). SADBE also causes more visible inflammation by day 14 (blue “*”). Robust hair growth is observed by day 17 on the treated flank.

### DETAILED DESCRIPTION

Compositions for treating hair-loss are not well-developed; currently available methods of treatment involve administration of compounds with little success and which cause discomfort to the patients.

### Hair Growth Compositions

As described herein, a number of hapten derivative compounds were developed. These compounds increase or stimulate hair growth. Some of the compounds increase hair growth while minimizing inflammation to the dermis. A compound of formula (1) was synthesized:

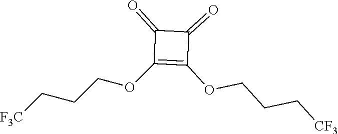 [SMILES: O=c1c(OCCCC(F)(F)F)c(OCCCC(F)(F)F)c1=O]

Subsequently, topical delivery of the present compounds demonstrated accelerated hair growth in mice, as compared to vehicle-control treated contralateral skin of the mice, all after prior shaving. In some cases, topical delivery of the novel compounds (e.g., CF3-SADBE) also demonstrated less inflammation than known treatments. Further, CF3-SADBE also appeared to demonstrate increased efficacy as measured by earlier induction of anagen upon topical delivery.

A non-limiting list of hapten derivatives that were developed and are useful herein include the following compounds:

| Name | Structure | MWt |
|---|---|---|
| MGH- CS1 | 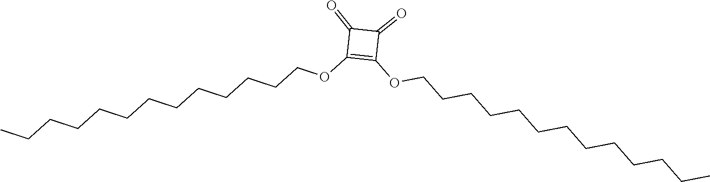 [SMILES: CCCCCCCCCCCCCOc1c(OCCCCCCCCCCCCC)c(=O)c1=O] | 478.7 |
| MGH- CS2 | 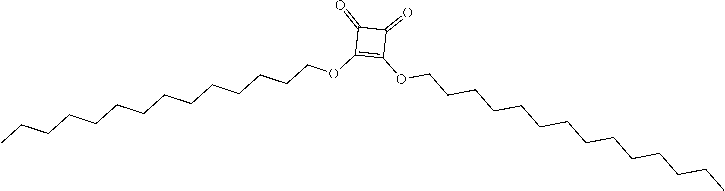 [SMILES: CCCCCCCCCCCCCCOc1c(OCCCCCCCCCCCCCC)c(=O)c1=O] | 506.8 |
| MGH- CS3 | 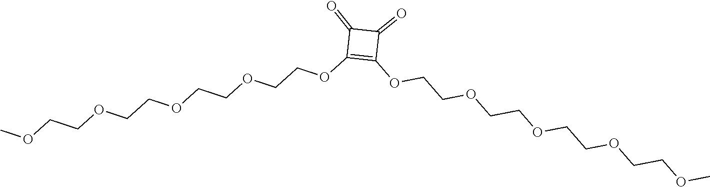 | 494.5 |
| MGH- CS4 | 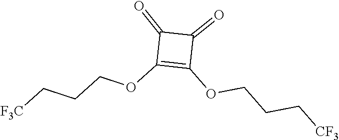 [SMILES: O=c1c(OCCCC(F)(F)F)c(OCCCC(F)(F)F)c1=O] | 334.2 |
| MGH- CS5 | 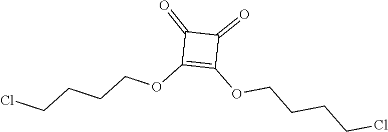 [SMILES: O=c1c(OCCCCCl)c(OCCCCCl)c1=O] | 294.1 |

To improve the therapeutic activities and safety of the topical hair growth agents, the present hapten derivative compounds were designed. In an attempt to improve solubility and possible skin penetration properties, PEG or saturated long alkyl chains were introduced to replace the dibutyl ester moieties. However, both compounds were not active at promoting hair growth in the animal models.

Shorter alkyl chains were then introduced. CF3 or CL substituted alkyl chains were designed. It was speculated that both compounds might have similar cross-linking reactivities. Surprisingly, the CF3-substituted compounds were active at promoting hair growth while the CL-substituted compounds showed low activity. This demonstrated that the hair growth promoting activity was not simply linked to the compound's reactivity as a crosslinking agent.

The CF3-compound (e.g., CF3-SADBE or MGH-CS-4 above) demonstrated accelerated hair growth relative to known treatments, as well as apparently somewhat diminished inflammation.

### Synthesis of CF3-SADBE

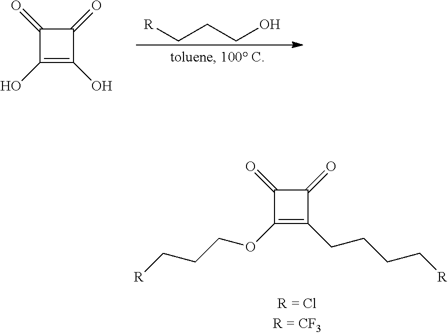

A solution of squaric acid (9 mmol) and alcohol (36 mmol) in toluene (9 ml) was refluxed at 100° C. using Dean-Stork apparatus for 16 h. After completion of the reaction toluene was concentrated in vacuo and resulting solution was diluted with ethyl acetate. The organic layer was washed with saturated sodium bicarbonate solution flowed by brine. The volatile were concentrated in vacuo and purified by column chromatography to yield respective compound.

### Use of Compositions for Stimulating Hair Growth

The compounds described herein can be used in compositions for treating hair loss, increasing hair growth, reducing hair loss, stimulating hair growth, thickening hair, or reducing the thinning of hair. Hair loss can be caused by a variety of conditions. Some of these conditions are associated with auto-immune diseases and some conditions are not auto-immune associated.

### Use of CF3-SADBE and Other Novel Compounds for Increasing Hair Growth

As demonstrated herein, CF3-SADBE can be used in the treatment of hair loss and/or increasing hair growth on a subject in need thereof. CF3-SADBE can be useful in the treatment of alopecia that is either associated with an autoimmune disease (e.g., alopecia areata) or alopecia that is not associated with an autoimmune disease. A non-limiting list of alopecia conditions (autoimmune associate alopecia and non-autoimmune alopecia) includes acne keloidalis (e.g., folliculitis keloidalis, acne keloidalis nuchae), alopecia areata (auto-immune alopecia; e.g., ophiasis), alopecia totalis, alopecia universalis, anagen effluvium, androgenetic alopecia (e.g., male and female pattern hair loss), Brocq's alopecia, central centrifugal cicatricial alopecia, cicatricial (scarring) alopecias (e.g., primary cicatricial alopecia), congenital alopecia, congenital atrichia, diffuse alopecia areata, discoid (lesions) lupus erythematosus (DLE), dissecting cellulitis (e.g., perifolliculitis, abscedens et suffodiens), “end-stage” or “burnt-out”cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris (e.g., diffuse), lipedematous alopecia (lipedematous scalp), male pattern hair loss (e.g., bitemporal), non-cicatricial (non-scarring) alopecias, psoriasis, psoriasiform alopecia, pressure (postoperative) alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis (see, e.g., Bolognia, et. al., Dermatology. Elsevier Health Sciences, Jun. 8, 2012. Pages 1163-1187. Section 11: Hair, Nails, and Mucous Membranes. 69: Alopecias). Methods for diagnosing these conditions, or for identifying subjects with these conditions, are known in the art; see, e.g., Bolognia, et. al., Dermatology. Elsevier Health Sciences, Jun. 8, 2012. Pages 1163-1187. Section 11: Hair, Nails, and Mucous Membranes. 69: Alopecias.

### Use of SADBE or DPCP for Treatment Alopecia beyond Autoimmune Alopecia

As described herein, surprisingly, the compounds SADBE and DPCP can also be used to treat alopecia that is not associated with an autoimmune disorder (e.g., for the treatment of alopecia conditions other than alopecia areata). SADBE and DPCP have been used for the treatment of alopecia areata, an autoimmune related alopecia. Before the present disclosure, however, it was not known that DPCP and/or SADBE could increase hair growth in a mammal without an autoimmune disease. As demonstrated herein, SADBE increased (e.g., promoted) hair growth in a mammal without an autoimmune disease. Also, DPCP demonstrated increased hair growth in a mammal without an autoimmune disease.

A non-limiting list of non-autoimmune related alopecia conditions includes acne keloidalis (e.g., folliculitis keloidalis, acne keloidalis nuchae), alopecia totalis, alopecia universalis, anagen effluvium, androgenetic alopecia (e.g., male and female pattern hair loss), Brocq's alopecia, central centrifugal cicatricial alopecia, cicatricial (scarring) alopecias (e.g., primary cicatricial alopecia), congenital alopecia, congenital atrichia, diffuse alopecia areata, discoid (lesions) lupus erythematosus (DLE), dissecting cellulitis (e.g., perifolliculitis, abscedens et suffodiens), “end-stage” or “burnt-out” cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris (e.g., diffuse), lipedematous alopecia (lipedematous scalp), male pattern hair loss (e.g., bitemporal), non-cicatricial (non-scarring) alopecias, psoriasis, psoriasiform alopecia, pressure (postoperative) alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis. Methods for diagnosing these conditions, or for identifying subjects with these conditions, are known in the art; see, e.g., Bolognia, et. al., Dermatology. Elsevier Health Sciences, Jun. 8, 2012. Pages 1163-1187. Section 11: Hair, Nails, and Mucous Membranes. 69: Alopecias.

### Combination Treatments

In some embodiments, the methods described herein can include administering a combination treatment. A non-limiting list of treatment options that can be administered with the compositions and methods described herein include topical, oral, and intralesional corticosteroids (e.g., clobetasol, fluocinonide), topical irritants (e.g., anthralin, tazarotene, azelalc acid), topical minoxidil, topical finasteride, dutasteride, topical immunotherapy, systemic corticosteroids (e.g., pulsed dosing), oral finasteride (e.g., a type II 5α-reductase inhibitor), oral tetracycline (e.g., doxycycline, minocycline), oral rifampin and clindamycin, TNF-α inhibitors, PPAR-γ agonists (e.g., pioglitazone hydrochloride), isotretinoin, oral zinc sulfate, oral antimalarial drugs (e.g., hydroxychloroquine), systemic JAK/STAT pathway inhibitors (e.g., tofactinib or ruxolitinib), topical or oral photochemotherapy, excimer laser, systemic corticosteroids (e.g., chronic), and systemic cyclosporine.

### Formulation and Administration of Compositions

The compositions of the disclosure can be utilized in many different ways. For example, they can be a component of a dry formulation or wet solution. They can be provided as a component of an injectable composition which is injected (e.g., injected intradermally or subcutaneously) into a balding area (e.g., the scalp). Alternatively, the compounds described herein can be a component of a composition be applied topically to a balding area or an area of hair-loss. This can be performed with the goal of thickening the appearance of the hair. These compositions can optionally be applied in combination with any known non-toxic delivery agent and/or penetrant.

The compositions of the disclosure can be administered topically or by injection. The dosage required depends on the choice of the route of administration; the nature of the formulation; the nature of the subject's condition; the subject's size, surface area of treatment, age, and sex; other drugs being administered; and the judgment of the attending physician. In some embodiments, suitable dosages are in the range of 0.01-500.0 mg/kg. Wide variations in the needed dosage are to be expected in view of the differing efficiencies of various routes of administration. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more fold). The compositions of the disclosure can also be administered in a single topical treatment. The composition of the disclosure can be administered in multiple topical treatments. Multiple administrations can be daily, every other day, bi-weekly, weekly, every other week, monthly, or any combination thereof. Administration of the compositions described herein can occur over a day, week, month, year, or longer. Encapsulation of the compound in a suitable delivery vehicle (e.g., a cream, emulsion, aqueous solution, or solid) may increase the efficiency of delivery. The dosing required also depends on the variability of the skin. Difference dosages may be needed due to different skin thickness at the hair-bearing location, number of applications, type of skin, different indications (e.g., different causes of alopecia), and/or different tolerance by the subject.

The compositions of this application can be prepared for storage by mixing them with any one or more of a variety of pharmaceutically acceptable carriers, excipients, or stabilizers known in the art. Acceptable carriers, excipients, or stabilizers are nontoxic to recipients at the dosages and concentrations employed, and include: buffers, such as phosphate, citrate, and other non-toxic organic acids; antioxidants such as ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol, or sorbitol; salt-forming counterions such as sodium; and/or nonionic surfactants such as Tween, Pluronics, or PEG.

The compositions can be in any form suitable for application to the scalp and/or hair, e.g., solutions, suspensions, lotions, creams, gels, toners, sticks, pencils, sprays, aerosols, ointments, cleansing liquid washes and solid bars, shampoos and hair conditioners, pastes, foams, milks, poultices, water-oil bilayer compositions, water-oil powder trilayer compositions, serums, powders, mousses, shaving creams, wipes, strips, patches, hydrogels, film-forming products, single-use applicators, and the like. The composition can be a shampoo, hair conditioner, or hair lotion. The composition form may follow from the particular dermatologically acceptable carrier chosen, if present in the composition. The composition may be in the form of an aqueous, aqueous-alcoholic, or oily solution, a lotion- or serum-type dispersion, or an emulsion (e.g., a milk-type emulsion of liquid or semi-liquid consistency). The composition may be in the form of a suspension or emulsion with a soft consistency of the aqueous or anhydrous cream or gel type, or alternatively, of microcapsules or microparticles, or of vesicular dispersions of the ionic and/or non-ionic type. The composition may be anhydrous or aqueous.

The compositions described herein can include a dermatologically acceptable carrier (also referred to herein simply as a “carrier”) for the composition. The phrase “dermatologically acceptable carrier”, as used herein, means that the carrier is suitable for topical application to the hair/scalp, has good aesthetic properties, is compatible with the hair anti-aging agents in the composition, and will not cause any unreasonable safety or toxicity concerns. A suitable carrier is selected to yield a desired product form. Furthermore, the solubility or dispersibility of the components may dictate the form and character of the carrier. In some embodiments, the carrier is present at a level of from about 50 wt % to about 99 wt %, about 60 wt % to about 98 wt %, about 70 wt % to about 98 wt %, or, alternatively, from about 80 wt % to about 95 wt %, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present disclosure may comprise silicone oils, non-silicone oils such as hydrocarbon oils, esters, ethers, and the like, and mixtures thereof.

The aqueous phase comprises water, such as demineralized or distilled water, for example. Other acceptable carriers that may be used in the aqueous carrier include, but are not limited to alcohol compounds, such as ethanol. According to one embodiment, the composition comprises alcohol, dipropylene glycol, and/or water.

The compositions have a pH ranging from about 3.0 to about 10 (e.g., between about pH 4.0 and about pH 9.0, between about pH 5.0 and about pH 9.0, or between about pH 6.0 and about pH 8.0), which may be measured by taking a direct pH measurement using a standard hydrogen electrode of the composition at 25 degrees C. Accordingly, the pH of the composition may be within the range from about 6 to about 9, for example.

Emulsions may further comprise an emulsifier. The composition may comprise any suitable percentage of emulsifier to sufficiently emulsify the carrier. Suitable weight ranges include from about 0.1 wt % to about 10 wt % or about 0.2 wt % to about 5 wt % of an emulsifier, based on the weight of the composition. Emulsifiers may be nonionic, anionic, or cationic. Suitable emulsifiers are disclosed in, for example, U.S. Pat. Nos. 3,755,560 and 4,421,769, and McCutcheon's Detergents and Emulsifiers, North American Edition, pages 317-324 (1986), which are incorporated herein by reference in their entirety. Suitable emulsions may have a wide range of viscosities, depending on the desired product form. Non-limiting examples of emulsifiers include glyceryl stearate, polysorbate 60, and the PEG-6/PEG-32/glycerol stearate mixture sold under the name of Trefose® by Gattefosse. An emulsion may contain a fatty phase that may range from between about 5 wt % to about 80 wt % (e.g., between about 5 wt % to about 50 wt %) of the composition. Any of the emulsions described herein may contain one or more agents selected from the group of oils, waxes, emulsifiers, and co-emulsifiers. Examples of oils, waxes, emulsifiers, and co-emulsifiers used in hair care compositions are well-known in the art. An emulsifier and a co-emulsifier may be present in the composition in a proportion ranging from 0.3 wt % to about 30 wt % (e.g., between about 0.5 wt % to about 20 wt %) of the composition. An emulsion may contain lipid vesicles.

The compositions (e.g., any of the compositions described herein) can include one or more (e.g., two, three, four, five, six, seven, eight, nine, ten, eleven, or twelve) of a hydrophilic gelling agent (e.g., carboxyvinyl polymers, acrylic copolymers (e.g., acrylate/alkyl acrylate copolymers), polyacrylamides, polysaccharides (e.g., hydroxypropylcellulose), natural gums, and clays), lipophilic gelling agent (e.g., modified clays (e.g., betones), metal salts of fatty acids (e.g., aluminum stearates), and hydrophobic silica, ethylcellulose, and polyethylene), hydrophilic or lipophilic additives, preservatives, antioxidants, solvents (e.g., ethanol, isopropanol, and propylene glycol), perfumes, fillers, odor absorbers, a dye, an oil (e.g., a mineral oil (e.g., liquid paraffin), a vegetable oil (e.g., shea butter or sunflower oil), an animal oil (e.g., perhydrosqualene), a synthetic oil (e.g., purcellin oil), or a silicone oil), a wax (e.g., cyclomethicone, beeswax, carnauba wax, or paraffin wax), a surfactant, an antiseptic, a metal ion chelating agent, a water-soluble polymer, a thickener, a pigment, an ultraviolet protectant, a moisturizer, an antioxidant, a pH adjuster, a cleansing agent, and a drying agent, e.g., with each of the one or more agents present in the composition between, e.g., about 0.01 wt % to about 10 wt % of the composition.

Any of the compositions provided herein can contain at least one preservative and/or dye.

The term “subject” or “patient” is defined herein to include animals. In some embodiments, the animal is a mammal, including, but not limited to, primates (e.g., humans), cows, sheep, goats, horses, dogs, cats, rabbits, rats, mice and the like. In some embodiments, the subject is a human. In some embodiments, the subject is a child.

### Use of Compounds in Cosmetic Compositions

The present disclosure is based, at least in part, on the development of cosmetic compositions that can be useful in stimulating, increasing, thickening, or accelerating hair growth. These cosmetic compositions could be used in subjects with alopecia or without alopecia. As described herein, hair growth can be stimulated, increased, thickened, or accelerated, e.g., temporarily, by applying to the scalp and/or hair follicles cosmetically effective amounts of the compositions and formulations described herein. As described herein, the loss of hair can be reduced, inhibited, delayed, or treated by applying to the scalp and/or hair follicles cosmetically effective amounts of the compositions and formulations described herein.

In some embodiments, the present disclosure provides for a cosmetic composition comprising one or more of the compounds described herein and a pharmaceutically acceptable carrier. In some cases, the cosmetic compositions also comprise one or more anti-inflammatory agents. The anti-inflammatory agents can be glucocorticoid agents, calcipotriol agents, antihistamines, and/or other immunosuppressive agents. The compositions described herein can also include any hair growth agents known in the art, including, e.g., minoxidil or finasteride.

The cosmetic compositions described herein can be formulated for topical or subcutaneous administration.

In some embodiments, the present disclosure provides for a cosmetic composition for increasing hair growth in a mammal in need thereof comprising an effective amount of one or more of the compounds described herein and a pharmaceutically or dermatologically acceptable carrier. Also described herein are methods for increasing hair growth, the method comprising topically applying an effective amount of a formulation to hair follicles and/or to the skin overlying hair follicles one or more times, wherein the formulation comprises one or more of the compounds described herein and a cosmetically acceptable carrier. The cosmetically acceptable carrier can comprises one or more of an aqueous gel, alcoholic gel, ointment, oil, alcoholic or aqueous fluid, water-in-oil emulsion, oil-in-water emulsion, and water-in-silicone emulsion. In some cases, the compositions described herein also include a cosmetically acceptable accessory ingredients selected from the group consisting of xanthan gum, glycerin, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance, and water.

### EXAMPLES

The compositions and methods of the present disclosure are further described in the following examples, which do not limit the scope of the disclosure described in the claims.

### Example 1: Topical Administration of SADBE and DPCP

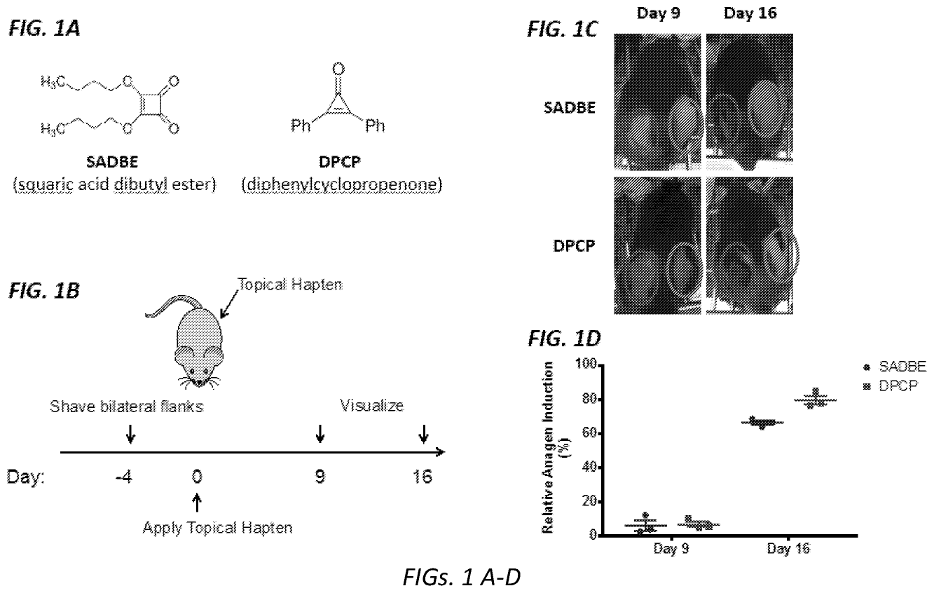

To measure the effect of SABDE and DPCP (see FIG. 1a) on hair growth, 6 female C57BL/6 mice, 8 weeks old, were shaved on both flanks 4 days before topical administration of the compounds (Day −4). During this period, the mice were visually monitored to confirm absence of hair growth and to confirm the entire shaved area is arrested in the telogen phase as expected from 8 weeks old mice. On the day of administration (day 0), 2% SABDE or 2% DCPC was topically administered to the shaved left flank of 3 mice each using a cotton-tipped applicator (see FIG. 1B). Images were taken on day 9 and day 16 in order to visualize hair growth and inflammation (see FIG. 1c). Skin pigmentation intensity was quantified by ImageJ. The anagen induction index is calculated as the change in skin pigmentation intensity (a marker of anagen) on the treated side relative to (i.e., as a percent of) the untreated side (see FIG. 1d).

The anagen induction index can be determined by any methods known in the art, including, for example, histological methods (e.g., H&E, other stains, or without stains) and/or other visual morphology methods. (e.g., methods to measure the biological parameters of hair growth. This value is then normalized to the specific experiment (individual mice) and shown as relative anagen induction by skin pigmentation intensity (a marker of anagen) on the treated side as a percent of the baseline untreated side. Analysis of the anagen (or other phases) can be performed by any method known in the art, including, e.g., a tissue biopsy and/or morphological analysis.

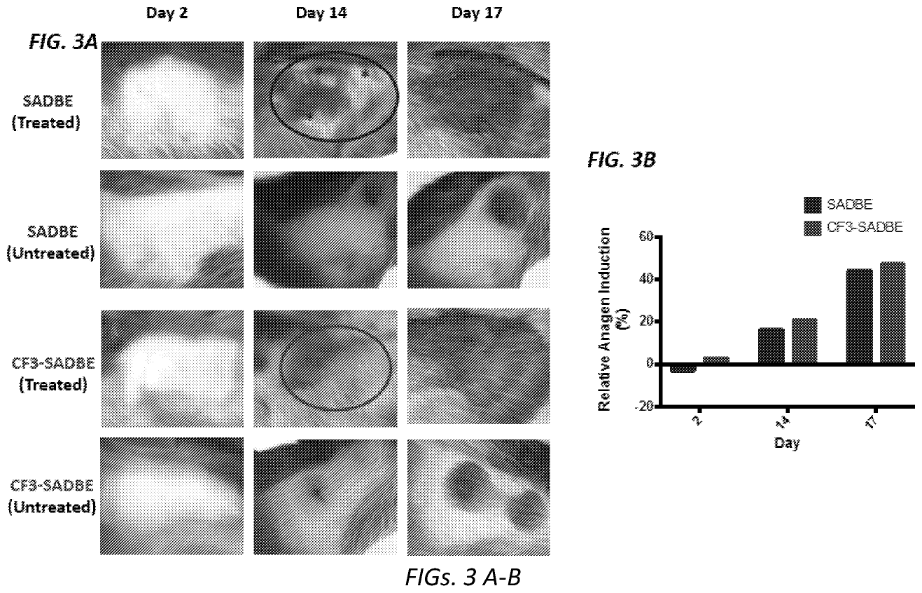

Inflammation can be measured by known methods such as histological grading, visual identification, and quantification of lesions. The number, size, and extent of the cutaneous lesions present (e.g., see blue * in FIG. 3a) can be visualized and quantified using any known method in the art, including, for example, histological methods (e.g., H&E, other stains, or without stains) and/or other visual morphology methods.

### Example 2: Topical Administration of Novel Compounds

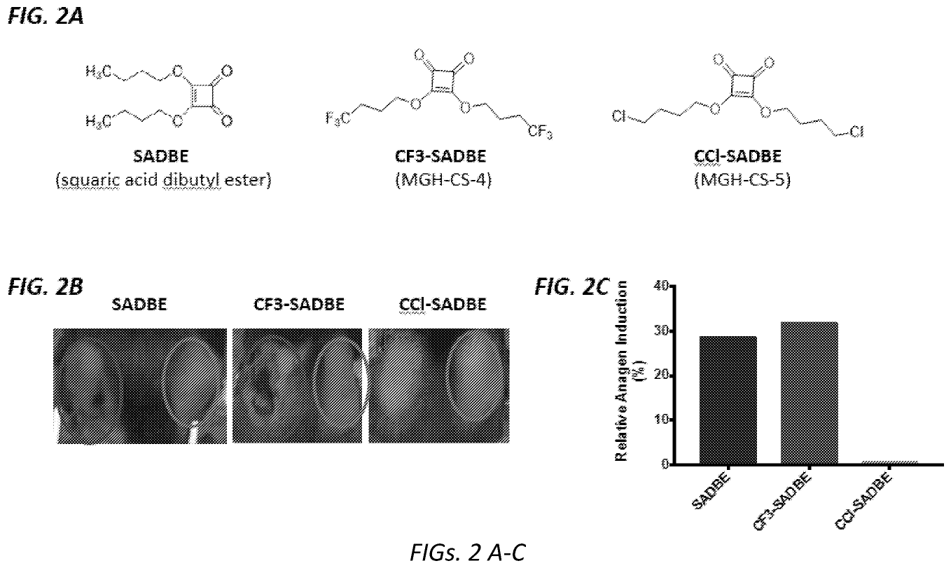

In order to compare the effect of SABDE, CF3-SABDE or CC1-SABDE (see FIG. 2a) on hair growth, 3 female C57BL/6 mice, 8 weeks old, were pre-sensitized with 2% of each compound on the chest 4 days before the first treatment and both flanks were shaved (Day 0). On days 3, 5, and 7, 2% of SABDE, CF3-SABDE or CC1-SABDE was topically administered to the shaved left flank of one mouse each using a cotton-tipped applicator. Images were taken on day 17 in order to visualize hair growth and inflammation (see FIGS. 2B and 2C).

### Example 3: Comparison of Known and Novel Compounds

The effects of a topical treatment dose of SADBE or CF3-SADBE were measured using the methods describe in Example 1. In FIG. 3, the topical treatment dose used was 50 uL of 100 mM SADBE or CF3-SADBE.

### Example 4: Induction of Anagen Phase by CF3-SADBE

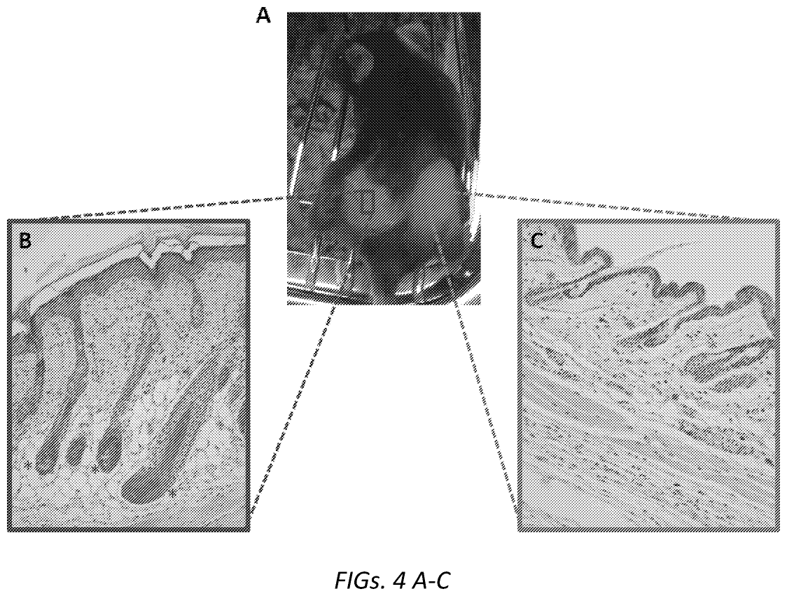

The effects of a topical treatment dose of CF3-SADBE was measured and visualized using the methods described in Example 1. The gross appearance of the mouse treated with CF3-SADBE was imaged (see FIG. 4a). CF3-SADBE was administered on the left flank at a dose of 100 uL of 100 mM CF3-SADBE (see FIG. 4B). The right flank was left untreated (see FIG. 4C).

### Example 5: Earlier Induction of Anagen by CF3-SADBE with Less Inflammation than SADBE

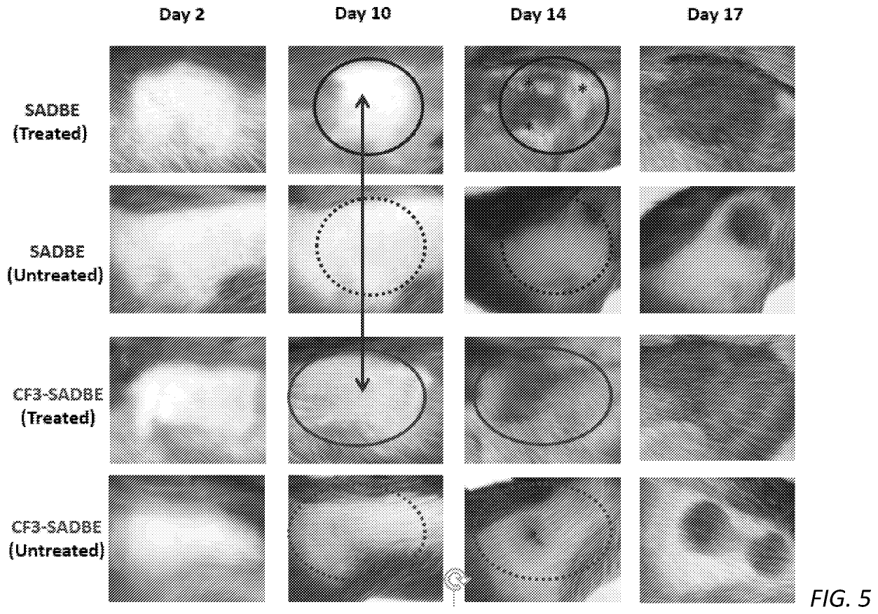

The effects on anagen induction and inflammation of topical treatment with CF3-SADBE at 100 mM were measured and visualized using the methods described in Example 1. Treatment with 100 mM CF3-SADBE induced visible anagen by day 10. Treatment with 100 mM SADBE did not induce visible anagen by day 10 (see purple arrow in FIG. 5). This demonstrates improved efficacy of CF3-SADBE. SADBE also caused more visible inflammation by day 14 (see FIG. 5 blue “*”). Robust hair growth was observed by day 17 on the treated flank.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A compound of formula (1):

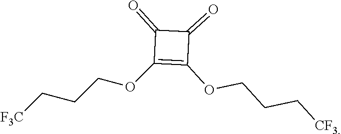 [SMILES: O=c1c(OCCCC(F)(F)F)c(OCCCC(F)(F)F)c1=O]

2. A pharmaceutical composition comprising the compound of claim 1, and a pharmaceutically acceptable carrier.

3. The pharmaceutical composition of claim 2, further comprising one or more anti-inflammatory agents.

4. The pharmaceutical composition of claim 3, wherein the anti-inflammatory agent is selected from the group consisting of glucocorticoid agents, calcipotriol, an immunosuppressive agent, and an antihistamine.

5. The pharmaceutical composition of claim 2, further comprising an agent selected from minoxidil, finasteride, SADBE, DPCP, and combinations thereof.

6. The pharmaceutical composition of claim 2, wherein the composition is formulated for topical or subcutaneous administration.

7. A pharmaceutical composition for increasing hair growth in a mammal in need thereof comprising an effective amount of the compound of claim 1 and a pharmaceutically or dermatologically acceptable carrier.

8. A hair care composition for increasing hair growth in a mammal in need thereof comprising an effective amount of the compound in claim 1 and a dermatologically acceptable carrier.

9. A method of increasing percentage of hair follicles in anagen phase and decreased percentage of hair follicles in telogen phase in a subject, the method comprising administering to a subject in need thereof an effective amount of the compound of claim 1.

10. The method of claim 9, wherein the subject has alopecia.

11. The method of claim 10, wherein the alopecia is selected from the group consisting of acne keloidalis, alopecia areata, alopecia totalis, alopecia universalis, anagen effluvium, androgenetic alopecia, Brocq's alopecia, central centrifugal cicatricial alopecia, cicatricial alopecias, congenital alopecia, congenital atrichia, diffuse alopecia areata, discoid lupus erythematosus (DLE), dissecting cellulitis, “end-stage” or “burnt-out” cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris, lipedematous alopecia, male pattern hair loss, non-cicatricial alopecias, psoriasis, psoriasiform alopecia, pressure alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis.

12. A method of treating hair loss in a subject, the method comprising administering a therapeutically effective amount of the compound of claim lto a subject in need thereof.

13. The method of claim 12, wherein the hair loss is associated with alopecia.

14. The method of claim 12, wherein the compound is administered to the skin, hair, or scalp of a mammal in need thereof.

15. A method of stimulating hair growth comprising administering a therapeutically effective amount of a compound of claim 1.

16. A method of increasing hair growth comprising administering a therapeutically effective amount of a compound of claim 1.

17. A method of inhibiting hair loss comprising administering a therapeutically effective amount of a compound of claim 1.

18. A method for increasing hair growth in a subject, the method comprising topically applying an effective amount of a formulation to hair follicles and/or to skin overlying hair follicles of the subject, wherein the formulation comprises the compound of claim 1 and an acceptable carrier, and the formulation is applied one, two, three, or more times.

19. The method of claim 18, wherein the acceptable carrier comprises one or more of an aqueous gel, alcoholic gel, ointment, oil, alcoholic or aqueous fluid, water-in-oil emulsion, oil-in-water emulsion, and water-in-silicone emulsion.

20. The method of claim 18, wherein the formulation further comprises an acceptable accessory ingredient selected from the group consisting of xanthan gum, glycerin, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance, and water.

21. The method of claim 18, wherein the formulation further comprises an agent selected from the group consisting of minoxidil, finasteride, DPCP, SADBE, and combinations thereof.

22. The method of claim 18, wherein the method increases hair growth by at least approximately 10%.

23. The method of claim 21, wherein the method increases hair growth by at least approximately 15%.

24. The method of claim 21, wherein the method increases hair growth by at least approximately 20%.

25. The method of claim 18, further comprising administering one or more steroids.

26. The method of claim 18, further comprising administering one or more agents to decrease sensitivity of the scalp or skin of the mammal in need thereof.

27. The method of claim 18, further comprising administering one or more agents to modulate inflammation.

28. A method of promoting transition of hair follicles into anagen phase, the method comprising administering a composition comprising an effective amount of compound 1.

29. A method of thickening hair, comprising administering a composition comprising an effective amount of compound 1.

30. A method of reducing hair thinning, comprising administering a composition comprising an effective amount of compound 1.

31. A pharmaceutical composition for increasing hair growth in a mammal in need thereof comprising an effective amount of SADBE and/or DPCP and a pharmaceutically or dermatologically acceptable carrier, wherein the mammal does not have an autoimmune disease.

32. A method of promoting transition of hair follicles from telogen phase to anagen phase, the method comprising administering to a subject in need thereof an effective amount of SADBE and/or DPCP, wherein the subject does not have an autoimmune disease.

33. The method of claim 32, wherein the subject has alopecia.

34. The method of claim 33, wherein the alopecia is selected from the group consisting of acne keloidalis, alopecia totalis, alopecia universalis, anagen effluvium, androgenetic alopecia, Brocq's alopecia, central centrifugal cicatricial alopecia, cicatricial alopecias, congenital alopecia, congenital atrichia, diffuse alopecia areata, discoid lupus erythematosus (DLE), dissecting cellulitis, “end-stage” or “burnt-out” cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris, lipedematous alopecia, male pattern hair loss, non-cicatricial alopecias, psoriasis, psoriasiform alopecia, pressure alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis.

35. A method of treating hair loss in a subject, the method comprising administering a therapeutically effective amount of SADBE and/or DPCP, wherein the hair loss is not associated with an autoimmune disorder.

36. The method of claim 35, wherein the hair loss is associated with non-autoimmune alopecia.

37. The method of claim 36, wherein the non-autoimmune alopecia is selected from the group consisting of acne keloidalis, alopecia totalis, alopecia universalis, anagen effluvium, androgenetic alopecia, Brocq's alopecia, central centrifugal cicatricial alopecia, cicatricial alopecias, congenital alopecia, congenital atrichia, diffuse alopecia areata, discoid lupus erythematosus (DLE), dissecting cellulitis, “end-stage” or “burnt-out” cicatricial alopecia, female pattern hair loss, folliculitis decalvans, frontal fibrosing alopecia, hair-shaft abnormalities, hyperandrogenism, hypotrichosis, inherited alopecia, lichen planus, lichen planopilaris, lipedematous alopecia, male pattern hair loss, non-cicatricial alopecias, psoriasis, psoriasiform alopecia, pressure alopecia, seborrheic dermatitis psoriasis, telogen effluvium, temporal triangular alopecia, tinea capitis, TNF-α inhibitor-induced psoriasiform alopecia, traction alopecia, trichorrhexis nodosa, trichotillomania, and tufted folliculitis.

38. The method of claim 35, wherein the compound is administered to the skin, hair or scalp of a mammal in need thereof.

39. A method of stimulating hair growth in a subject in need thereof, the method comprising administering a therapeutically effective amount of a SADBE and/or DPCP, wherein the subject does not have an autoimmune disease.

40. A method of increasing hair growth in a subject in need thereof, the method comprising administering a therapeutically effective amount of a SADBE and/or DPCP, wherein the subject does not have hair loss associated with an autoimmune disease.

41. A method of inhibiting hair loss in a subject in need thereof, the method comprising administering a therapeutically effective amount of a SADBE and/or DPCP, wherein the subject does not have hair loss associated with an autoimmune disease.

42. A method of increasing hair growth in a subject in need thereof, the method comprising topically applying an effective amount of a formulation to hair follicles and/or to the skin overlying hair follicles, wherein the formulation comprises SADBE and/or DPCP and an acceptable carrier, the formulation is applied one, two, three, or more times, and the subject does not have hair loss associated with an autoimmune disease.

43. The method of claim 42, wherein the acceptable carrier comprises one or more of an aqueous gel, alcoholic gel, ointment, oil, alcoholic or aqueous fluid, water-in-oil emulsion, oil-in-water emulsion, and water-in-silicone emulsion.

44. The method of claim 42, wherein the formulation further comprises an acceptable accessory ingredient selected from the group consisting of xanthan gum, glycerin, EDTA, sodium benzoate, phenoxyethanol, 2-hydroxy fatty alcohol alkoxylate, sodium polyacrylate, polysorbate 20, BHT, disaccharidic gums, ethylhexylglycerin, carbomer, butyleneglycol, acrylate polymers, PEG-40 hydrogenated castor oil, methylisothiazolinone, methylchloroisothiazolinone, propylene glycol, potassium sorbate, polyglyceryl caprylate, fragrance, and water.

45. The method of claim 42, wherein the formulation comprises an agent selected from the group consisting of minoxidil, finasteride, DPCP, SADBE, and combinations thereof.

46. The method of claim 42, wherein the method increased hair growth by at least approximately 10%.

47. The method of claim 42, wherein the method increased hair growth by at least approximately 15%.

48. The method of claim 42, wherein the method increased hair growth by at least approximately 20%.

49. The method of claim 42, further comprising administering one or more steroids.

50. The method of claim 42, further comprising administering one or more agents to decrease sensitivity of the scalp or skin of the mammal in need thereof.

51. The method of claim 42, further comprising administering one or more agents to modulate inflammation.

52. A method of promoting transition of hair follicles from telogen phase into anagen phase in a subject in need thereof, the method comprising administering a composition comprising an effective amount of SADBE or DPCP, wherein the subject does not have hair loss associated with an autoimmune disease.

53. A method of thickening hair in a subject in need thereof, the method comprising administering a composition comprising an effective amount of SADBE or DPCP, wherein the subject does not have hair loss associated with an autoimmune disease.

54. A method of reducing hair thinning in a subject in need thereof, the method comprising administering a composition comprising an effective amount of SADBE or DPCP, wherein the subject does not have hair loss associated with an autoimmune disease.
